(19) United States
(12) Patent Application Publication
(10) Pub. No.: US 2026/0263733 A1
(43) Pub. Date: Sep. 10, 2026

(54) MOOD STATE

(71) Applicant: Nicoventures Trading Limited, London (GB)

(72) Inventors: Jeffrey Sean SMITH, London (GB); Sean LUKAN, London (GB); Laura SMITH, London (GB); Chloe CORDERY, London (GB)

(21) Appl. No.: 19/165,234

(22) PCT Filed: Mar. 15, 2024
(86) PCT No.: PCT/GB2024/050698

§ 371 Date: Sep. 15, 2025

Related U.S. Application Data
(63) Continuation of application No. 18/184,203, filed on Mar. 15, 2023, now abandoned.

(51) Int. Cl. A61M 21/02; A61M 21/00; G06F 3/01

(52) U.S. Cl. CPC A61M 21/02; G06F 3/015; A61M 2021/0077; A61M 2205/3306; A61M 2205/3584; A61M 2209/088

(57) ABSTRACT

An apparatus, method and computer program is described comprising: communicating with one or more sensors generating real-time physiological data relating to a user within a room; determining delivery of one or more active compounds to influence, manage or control the mood state of said user; and communicating with one or more dispensers for delivery of said one or more active compounds into the room.

### FIELD

The present specification relates to management or control of a mood state of user.

### BACKGROUND

Many systems are known for delivering one or more active compounds to a user that seek to change or control a mood state of the user. There remains a need for further developments in this field.

### SUMMARY

In a first aspect, this specification describes a system comprising: an input module for communicating with one or more sensors generating real-time physiological data relating to a user within a room; a control module for determining delivery of one or more active compounds to influence, manage or control the mood state of said user; and an output module for communicating with one or more dispensers (e.g. a plurality of dispensers) for delivery of said one or more active compounds into the room.

The system may further comprise a prediction module for determining a future mood state or need of a user based, at least in part, on said real-time physiological data, wherein said control module determines said delivery of one or more active compounds based, at least in part, on the determined future mood state or need.

The system may further comprise said one or more dispensers. Alternatively, or in addition, the system may further comprise said one or more sensors.

The one or more dispensers may comprise one or more active dispensers; one or more lightbulbs and/or one or more light fittings.

The one or more sensors may comprise Internet of Things (IoT) devices, imaging devices and/or cameras. Some or all of said sensors may be remote from said user. Alternatively, or in addition, some or all of said sensors are in wireless communication with said system.

In some example embodiments, some or all of said active compounds are delivered as aerosols.

In the use of said system, the user may be in a virtual world (e.g. the Metaverse).

The system may further comprise a headset (e.g. a VR, AR, MR or similar headset) to be worn by said user. Some or all of said one or more sensors may be remote from said headset. Alternatively, or in addition, some or all of said one or more sensors may be provided by the headset. Some or all of said dispensers may be remote from said headset. Alternatively, or in addition, some or all of said dispensers may be provided by the headset.

In a second aspect, this specification describes a method comprising: communicating with one or more sensors generating real-time physiological data relating to a user within a room; determining delivery of one or more active compounds to influence, manage or control the mood state of said user; and communicating with one or more dispensers (e.g. a plurality if dispensers) for delivery of said one or more active compounds into the room. The user may be in a virtual world (e.g. the Metaverse) and/or wearing a headset.

The method may further comprise determining a future mood state of a user based, at least in part, on said real-time physiological data, wherein said control module determines said delivery of one or more active compounds based, at least in part, on the determined future mood state or need.

Some or all of said active compounds may be delivered as aerosols.

Some or all of said sensors may be remote from said user.

Some or all of said sensors may be in wireless communication with said system.

Some or all of said sensors may be remote from a virtual reality headset to be worn by said user.

In a third aspect, this specification describes computer-readable instructions which, when executed by a computing apparatus, cause the computing apparatus to perform (at least) any method as described herein (including the method of the second aspect described above).

In a fourth aspect, this specification describes a computer-readable medium (such as a non-transitory computer-readable medium) comprising program instructions stored thereon for performing (at least) any method as described herein (including the method of the second aspect described above).

In a fifth aspect, this specification describes an apparatus comprising: at least one processor; and at least one memory including computer program code which, when executed by the at least one processor, causes the apparatus to perform (at least) any method as described herein (including the method of the second aspect described above).

In a sixth aspect, this specification describes a computer program comprising instructions for causing an apparatus to perform at least the following: communicating with one or more sensors generating real-time physiological data relating to a user within a room; determining delivery of one or more active compounds to influence, manage or control the mood state of said user; and communicating with one or more dispensers for delivery of said one or more active compounds into the room.

### BRIEF DESCRIPTION OF THE DRAWINGS

Example embodiments will now be described, by way of example only, with reference to the following schematic drawings, in which:

FIG. 1 is a block diagram of a system in accordance with an example embodiment;

FIG. 2 is a block diagram of an example implementation of the delivery mechanism of the system of FIG. 1, in accordance with an example embodiment;

FIG. 3 is a flow chart showing an algorithm in accordance with an example embodiment;

FIG. 4 is a block diagram of an example implementation of the control module of the system of FIG. 1, in accordance with an example embodiment;

FIG. 5 is a flow chart showing an algorithm in accordance with an example embodiment;

FIG. 6 is a block diagram demonstrating example types of immersion in accordance with an example embodiment.

FIG. 7 is a block diagram showing an aerosol delivery mechanism in accordance with an example embodiment;

FIGS. 8 to 11 show users wearing headsets and aerosol delivery mechanisms in accordance with example embodiments;

FIG. 12 is a block diagram of an example implementation of the delivery mechanism of the system of FIG. 1, in accordance with an example embodiment;

FIGS. 13 to 15 show aerosol delivery mechanisms in accordance with example embodiments;

FIG. 16 is a block diagram of a system in accordance with an example embodiment;

FIG. 17 shows a user in accordance with an example embodiment;

FIG. 18 shows a transdermal optical imaging sensor used in some example embodiments;

FIG. 19 is a block diagram of a neural network used in some example embodiments;

FIG. 20 is a block diagram showing an aerosol delivery mechanism in accordance with an example embodiment; and

FIG. 21 is a block diagram of a processing system that may be used to implement one or more of the example embodiments.

### DETAILED DESCRIPTION

As used herein, the term “delivery mechanism” is intended to encompass systems that deliver a substance to a user, and includes:
non-combustible aerosol provision systems that release compounds from an aerosolisable material without combusting the aerosolisable material, such as electronic cigarettes, tobacco heating products, and hybrid systems to generate aerosol using a combination of aerosolisable materials; and
articles comprising aerosolisable material and configured to be used in one of these non-combustible aerosol provision systems.

According to the present disclosure, a “non-combustible” aerosol provision system is one where a constituent aerosol-generating material of the aerosol provision system (or component thereof) is not combusted or burned in order to facilitate delivery of at least one substance to a user.

In some embodiments, the delivery system is a non-combustible aerosol provision system, such as a powered non-combustible aerosol provision system.

In some embodiments, the non-combustible aerosol provision system is an electronic cigarette, also known as a vaping device or electronic nicotine delivery system (END), although it is noted that the presence of nicotine in the aerosol-generating material is not a requirement.

In some embodiments, the non-combustible aerosol provision system is an aerosol-generating material heating system, also known as a heat-not-burn system. An example of such a system is a tobacco heating system.

In some embodiments, the non-combustible aerosol provision system is a hybrid system to generate aerosol using a combination of aerosol-generating materials, one or a plurality of which may be heated. Each of the aerosol-generating materials may be, for example, in the form of a solid, liquid or gel and may or may not contain nicotine. In some embodiments, the hybrid system comprises a liquid or gel aerosol-generating material and a solid aerosol-generating material. The solid aerosol-generating material may comprise, for example, tobacco or a non-tobacco product.

Typically, the non-combustible aerosol provision system may comprise a non-combustible aerosol provision device and a consumable for use with the non-combustible aerosol provision device.

In some embodiments, the disclosure relates to consumables comprising aerosol-generating material and configured to be used with non-combustible aerosol provision devices. These consumables are sometimes referred to as articles throughout the disclosure.

In some embodiments, the non-combustible aerosol provision system, such as a non-combustible aerosol provision device thereof, may comprise a power source and a controller. The power source may, for example, be an electric power source or an exothermic power source. In some embodiments, the exothermic power source comprises a carbon substrate which may be energised so as to distribute power in the form of heat to an aerosol-generating material or to a heat transfer material in proximity to the exothermic power source.

In some embodiments, the non-combustible aerosol provision system may comprise an area for receiving the consumable, an aerosol generator, an aerosol generation area, a housing, a mouthpiece, a filter and/or an aerosol-modifying agent.

In some embodiments, the consumable for use with the non-combustible aerosol provision device may comprise aerosol-generating material, an aerosol-generating material storage area, an aerosol-generating material transfer component, an aerosol generator, an aerosol generation area, a housing, a wrapper, a filter, a mouthpiece, and/or an aerosol-modifying agent.

In some embodiments, the substance to be delivered may be an aerosol-generating material or a material that is not intended to be aerosolised. As appropriate, either material may comprise one or more active constituents, one or more flavours, one or more aerosol-former materials, and/or one or more other functional materials.

In some embodiments, the substance to be delivered comprises an active substance (sometimes referred to herein as an active compound).

The active substance as used herein may be a physiologically active material, which is a material intended to achieve or enhance a physiological response. The active substance may for example be selected from nutraceuticals, nootropics, psychoactives, or digiceutical or other technical/electronic devices that may induce a physiological response, such as vagus nerve stimulation (VGS). The active substance may be naturally occurring or synthetically obtained. The active substance may comprise for example nicotine, caffeine, taurine, theine, vitamins such as B6 or B12 or C, melatonin, cannabinoids, or constituents, derivatives, or combinations thereof. The active substance may comprise one or more constituents, derivatives or extracts of tobacco, cannabis or another botanical. In one embodiment, the active substance is a legally permissible recreational drug.

In some embodiments, the active substance comprises nicotine. In some embodiments, the active substance comprises caffeine, melatonin or vitamin B12.

As noted herein, the active substance may comprise one or more constituents, derivatives or extracts of cannabis, such as one or more cannabinoids or terpenes.

As noted herein, the active substance may comprise or be derived from one or more botanicals or constituents, derivatives or extracts thereof. As used herein, the term “botanical” includes any material derived from plants including, but not limited to, extracts, leaves, bark, fibres, stems, roots, seeds, flowers, fruits, pollen, husk, shells or the like. Alternatively, the material may comprise an active compound naturally existing in a botanical, obtained synthetically. The material may be in the form of liquid, gas, solid, powder, dust, crushed particles, granules, pellets, shreds, strips, sheets, or the like. Example botanicals are tobacco, eucalyptus, star anise, hemp, cocoa, cannabis, fennel, lemongrass, peppermint, spearmint, rooibos, chamomile, flax, ginger, Ginkgo biloba, hazel, hibiscus, laurel, licorice (liquorice), matcha, mate, orange skin, papaya, rose, sage, tea such as green tea or black tea, thyme, clove, cinnamon, coffee, aniseed (anise), basil, bay leaves, cardamom, coriander, cumin, nutmeg, oregano, paprika, rosemary, saffron, lavender, lemon peel, mint, juniper, elderflower, vanilla, wintergreen, beefsteak plant, curcuma, turmeric, sandalwood, cilantro, bergamot, orange blossom, myrtle, cassis, valerian, pimento, mace, damien, marjoram, olive, lemon balm, lemon basil, chive, carvi, verbena, tarragon, geranium, mulberry, ginseng, theanine, theacrine, maca, ashwagandha, damiana, guarana, chlorophyll, baobab or any combination thereof. The mint may be chosen from the following mint varieties: Mentha arventis, Mentha c.v., Mentha niliaca, Mentha piperita, Mentha piperita citrata c.v., Mentha piperita c.v, Mentha spicata crispa, Mentha cardifolia, Mentha longifolia, Mentha suaveolens variegata, Mentha pulegium, Mentha spicata c.v. and Mentha suaveolens

In some embodiments, the active substance comprises or is derived from one or more botanicals or constituents, derivatives or extracts thereof and the botanical is tobacco.

In some embodiments, the active substance comprises or derived from one or more botanicals or constituents, derivatives or extracts thereof and the botanical is selected from eucalyptus, star anise, cocoa and hemp.

In some embodiments, the active substance comprises or derived from one or more botanicals or constituents, derivatives or extracts thereof and the botanical is selected from rooibos and fennel.

Aerosolisable material, which also may be referred to herein as aerosol generating material, is material that is capable of generating aerosol, for example when heated, irradiated or energized in any other way. Aerosolisable material may, for example, be in the form of a solid, liquid or gel which may or may not contain nicotine and/or flavourants.

The aerosol-generating material may be an “amorphous solid”. In some embodiments, the amorphous solid is a “monolithic solid”. The aerosol-generating material may be non-fibrous or fibrous. In some embodiments, the aerosol-generating material may be a dried gel. The aerosol-generating material may be a solid material that may retain some fluid, such as liquid, within it. In some embodiments the retained fluid may be water (such as water absorbed from the surroundings of the aerosol-generating material) or the retained fluid may be solvent (such as when the aerosol-generating material is formed from a slurry). In some embodiments, the solvent may be water.

The aerosol-generating material may comprise one or more active substances and/or flavours, one or more aerosol-former materials, and optionally one or more other functional material.

The aerosol-former material may comprise one or more constituents capable of forming an aerosol. In some embodiments, the aerosol-former material may comprise one or more of glycerine, glycerol, propylene glycol, diethylene glycol, triethylene glycol, tetraethylene glycol, 1,3-butylene glycol, erythritol, meso-Erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, and propylene carbonate.

The one or more other functional materials may comprise one or more of pH regulators, colouring agents, preservatives, binders, fillers, stabilizers, and/or antioxidants.

The material may be present on or in a support, to form a substrate. The support may, for example, be or comprise paper, card, paperboard, cardboard, reconstituted material, a plastics material, a ceramic material, a composite material, glass, a metal, or a metal alloy. In some embodiments, the support comprises a susceptor. In some embodiments, the susceptor is embedded within the material. In some alternative embodiments, the susceptor is on one or either side of the material.

A consumable is an article comprising or consisting of aerosol-generating material, part or all of which is intended to be consumed during use by a user. A consumable may comprise one or more other components, such as an aerosol-generating material storage area, an aerosol-generating material transfer component, an aerosol generation area, a housing, a wrapper, a mouthpiece, a filter and/or an aerosol-modifying agent. A consumable may also comprise an aerosol generator, such as a heater, that emits heat to cause the aerosol-generating material to generate aerosol in use. The heater may, for example, comprise combustible material, a material heatable by electrical conduction, or a susceptor.

An aerosol generator is an apparatus configured to cause aerosol to be generated from the aerosol-generating material. In some embodiments, the aerosol generator is a heater configured to subject the aerosol-generating material to heat energy, so as to release one or more volatiles from the aerosol-generating material to form an aerosol. In some embodiments, the aerosol generator is configured to cause an aerosol to be generated from the aerosol-generating material without heating. For example, the aerosol generator may be configured to subject the aerosol-generating material to one or more of vibration, increased pressure, or electrostatic energy.

A mood state of a person (or user) may depend on a number of environmental factors such as work, exercise, gaming or any activity. Example embodiments described herein relate to the control of delivery of active compounds for the purpose of managing, controlling or influencing mood states.

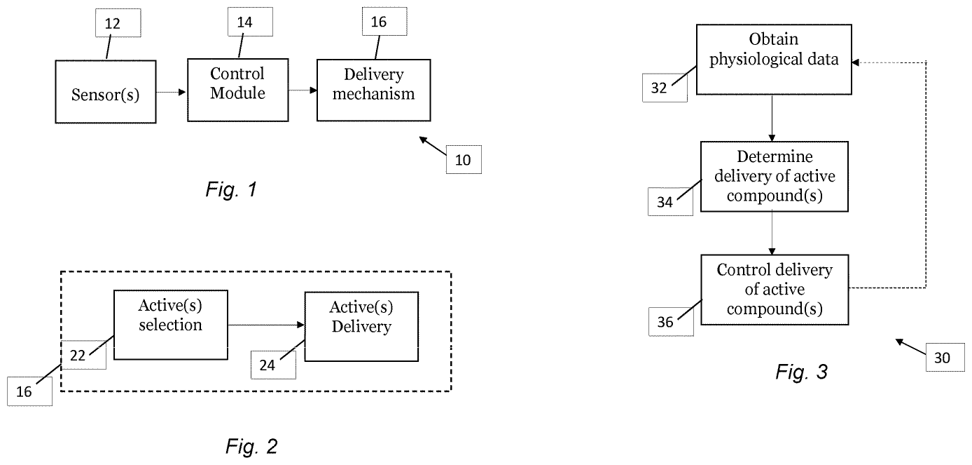

FIG. 1 is a block diagram of a system, indicated generally by the reference numeral 10, in accordance with an example embodiment. The system 10 comprises one or more sensors 12, a control module 14 and a delivery mechanism 16. It should be noted that in some example embodiments the sensors 12 provide inputs from outside the system 10 (rather than being a part of the system 10). Similarly, at least some features of the delivery mechanism 16 may be outside the system 10 (and may receive outputs from the system 10).

As discussed in detail below, the sensor(s) 12 provide real-time data (such as physiological data relating to a user) that are suitable for use in determining a mood state of a user. The control module 14 controls the delivery mechanism 16 in order to influence the user mood state. More specifically, the control module receives the sensors inputs and determine delivery of one or more active compounds (sometimes referred to herein as active substances) in order to influence, manage or control the mood state of the user.

The delivery mechanism 16 implements the determined delivery of said one or more active compounds. In some of the example embodiments discussed in detail below, the delivery mechanism 16 is configured to be worn by said user. Alternatively, or in addition, the delivery mechanism 16 may provide in-room delivery of active compounds. The delivery mechanism may deliver the one or more active compounds in the form of an aerosol, but this is not essential to all example embodiments; for example one or more active compounds may be delivered as a mist or a spray.

FIG. 2 is a block diagram of an example implementation of the delivery mechanism 16, in accordance with an example embodiment. As shown in FIG. 2, the example delivery mechanism 16 comprises an active(s) selection module 22 and an active(s) delivery module 24. The control module 14 of the system 10 is able to select one or more active compounds for delivery using the active(s) selection module 22 and to control the delivery of the selected active compound(s) using the active(s) delivery module 24.

FIG. 3 is a flow chart showing an algorithm, indicated generally by the reference numeral 30, in accordance with an example embodiment. The algorithm 30 may be implemented using the system 10 described above.

The algorithm 30 starts at step 32, where physiological data is obtained. The physiological data may be obtained from (or derived from) the sensor(s) 12 described above. The physiological data may comprise real-time physiological data (such as one or more of: ECG or EKG data, EEG data, temperature, oxygen usage, and eye movement data) suitable for use in determining a mood state of a user.

At operation 34, the control module 14 is used to determine parameters for delivery of one or more active compounds using the delivery mechanism 16.

In some example examples, the current mood state of a user is determined (based, at least in part, on the physiological data obtained in the operation 32) and used in the operation 34 to determine parameters for delivery of one or more active compounds. In other example embodiments, a future mood state or need of a user may be determined based, at least in part, on the physiological data obtained in the operation 32. The future mood state may be generated by a prediction module (discussed further below) that may form part of the control module 14. The active compound delivery determined in the operation 34 may be based, at least in part, on the determined future mood state.

At operation 36, delivery of the one or more active compounds to the user is controlled (based on output(s) from the control module 14). The operation 36 may include the use of the active(s) selection module 22 to determine/select one or more active compounds to be delivered to the user. The operation 36 may further include the use of the active(s) delivery module 24 to deliver the selected actives. For example, the active(s) delivery module 24 may determine timing of delivery of selected active compounds (e.g. a delivery state time and a delivery duration). Selection of active compound(s) may be based on a-priori knowledge of their effects on the user's mood. Some example delivery mechanisms are discussed below.

There are many active compounds, or combinations of active compounds, that may be delivered under the control of the algorithm 30. Examples include melatonin (e.g. to aid sleep), caffeine (e.g. to aid focus or alertness or to provide energy) and/or cannabidiol (CBD) (e.g. to aid relaxation). The skilled person will be aware of many other compounds (or combinations of compounds) that could be used (including other active compounds mentioned herein).

With the delivery of active compounds controlled, the algorithm 30 may return to operation 32, where further physiological data is obtained. Thus, the algorithm 30 may be iterative. Indeed, the algorithm 30 may enable the impact of the delivery of selected active compounds to be monitored and used to update the delivery of active compounds in the future. It should be noted that a delay or “wait state” may be provided in order to wait for an impact of a delivered compound to become apparent.

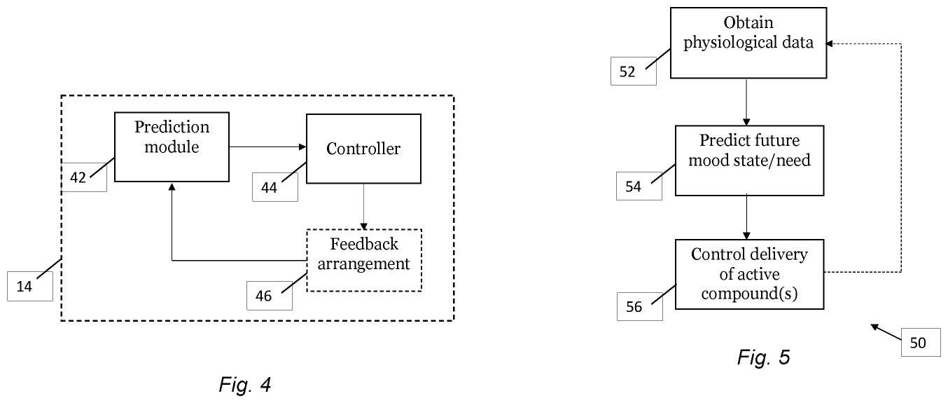

FIG. 4 is a block diagram of an example implementation of the control module 14 of the system of FIG. 1, in accordance with an example embodiment. As shown in FIG. 4, the example delivery mechanism 14 comprises a prediction module 42 for determining a future mood state of the user based, at least in part, on said real-time physiological data, and a controller 44 that controls the delivery of one or more active compounds based, at least in part, on the determined future mood state. The example control module 14 may further comprise a feedback arrangement 46 that enables the output of the prediction module 42 to be updated, for example based on the outcome of the delivery of active compound(s).

FIG. 5 is a flow chart showing an algorithm, indicated generally by the reference numeral 50, in accordance with an example embodiment. The algorithm 50 has many similarities with the algorithm 30 described above.

The algorithm 50 starts at step 52, where (as in the operation 32 described above) physiological data is obtained. The physiological data may be obtained from (or derived from) the sensor(s) 12 described above. The physiological data may be real-time physiological data suitable for use in determining a mood state of a user.

At operation 54, a future mood state or need of the user is determined based, at least in part, on the physiological data obtained in the operation 52. The future mood state or need may be generated by the prediction module 42 that may form part of the control module 14.

At operation 56, delivery of the one or more active compounds to the user is controlled based, at least in part, on the future mood state or need determined in the operation 54.

The operation 56 may include the use of the active(s) selection module 22 to determine/select one or more active compounds (such as one or more of the active compounds discussed above) to be delivered to the user and the active(s) delivery module 24 to deliver the selected actives. For example, the active(s) delivery module 24 may determine timing of delivery of selected active compounds (e.g. a delivery state time and a delivery duration). The active(s) selection module 22 may comprise a look up table providing the intended effect of each active compound available to the system such as stress relief etc. The look up table may further comprise details of the advised delivery time and delivery duration of each active compound in order to achieve the intended effect. The look up table may therefore be used in the selection of active compounds to meet a need and/or in determining the delivery parameters of a selected active compound to meet a need.

With the delivery of active compounds controlled, the algorithm 50 may return to operation 52, where further physiological data is obtained. Thus, the algorithm 50 may be iterative. Indeed, as with the algorithm 30 described above, the algorithm 50 may enable the impact of the delivery of selected active compounds to be monitored and used to update the delivery of active compounds in the future. It should be noted that a delay or “wait state” may be provided in order to wait for an impact of a delivered compound to become apparent.

The algorithms 30 and 50 may be used to influence, manage or control a mood state of a user who is within a virtual world. In some circumstances, influencing, managing or controlling the mood state of a user in a virtual world may enable an immersive experience of the user to be enhanced.

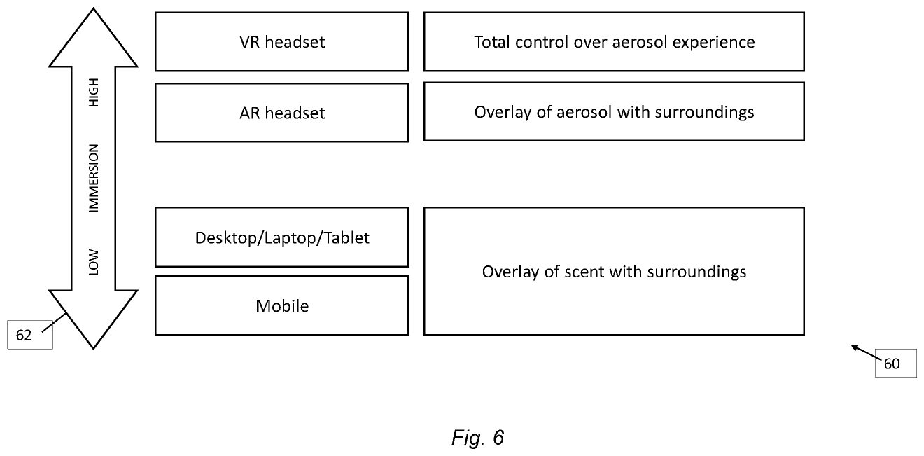

FIG. 6 is a block diagram, indicated generally by the reference numeral 60, demonstrating example types of immersion in accordance with an example embodiment.

The block diagram 60 includes a scale 62 indicating an immersion level of a user. The highest level of immersion may occur with the user wearing a virtual reality (VR) or similar headset. The use of virtual reality enables video content to be provided to a user using VR display system. The displayed content represents a VR space or world for immersive output through the display system. In some embodiments, audio is provided and a VR headset may be configured to provide VR video and audio content to the user, e.g. through the use of a pair of video screens and headphones incorporated within the headset.

Augmented reality (AR) refers to a real-world view that is augmented by computer-generated sensory input. Since the real-world view remains visible, the degree of immersion provided by AR is generally less than VR (as indicated by FIG. 6).

The provision of displays using a monitor of a computer (desktop, laptop or tablet) or a mobile device (e.g. a smartphone) offers a lower degree of immersion. Nevertheless, the management or control of mood can increase an overall immersive experience in such circumstances.

As discussed further below, the system 10 described above, or parts of that system (e.g. the delivery mechanism 16) may form part of a headset, such as a virtual reality, augmented reality, mixed reality or extended reality headset.

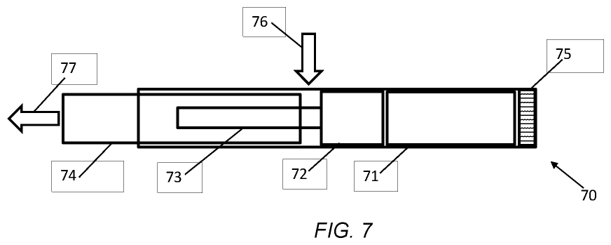

FIG. 7 is a block diagram of an aerosol generating device, indicated generally by the reference numeral 70, in accordance with an example embodiment. The aerosol generating device 70 may be used as, or form part of, the delivery mechanism 16 described above.

The aerosol generating device 70 comprises a battery 71, a control circuit 72, a heater 73 and a consumable 74. The device also includes a connector 75 (such as a USB connector). The connector 75 may enable connection to be made to a power source for charging the battery 71, for example under the control of the control circuit 72. The control circuit 72 may form part of (or being under the control of) the control module 14 described above.

In the use of the device 70, the heater 73 is inserted into the consumable 74, such that the consumable may be heated to generate an aerosol. In the use of the device 70, air is drawn into the device through an air inlet as indicated by arrow 76, then passes through the consumable, delivering the aerosol to the user as indicated by arrow 77.

The aerosol generating device 70 is provided by way of example only. Many alternative aerosol generating devices may be used in example implementations of the principles described here. For example, the aerosol generating device 70 may have access to multiple active compounds and include a mechanism for selecting between active compounds for delivery, as discussed further below. Such multiple active compounds could be mixed into bespoke formulations, for example based on past used experience.

Furthermore, the aerosol generating device 70 may be replaced with an alternative device for delivering active compounds in the form of a mist or spray. Other suitable arrangements will be apparent to those of ordinary skill in the art.

FIG. 8 shows a user 80 wearing a headset 82 (e.g. a VR, AR, MR or XR headset) and an aerosol delivery mechanism 84 in accordance with an example embodiment. The aerosol delivery mechanism 84 may be an inhaler.

The aerosol delivery mechanism 84 takes the form of a neck-wearable aerosol device. The headset 82 and aerosol delivery mechanism 84 allow the user 80 to connect to a virtual world (or metaverse). The headset 82 may provide visual and audio stimuli to the user 80 whilst the aerosol delivery mechanism 84 provides an aerosol (e.g. providing a smell and/or delivering an active compound). The virtual world can thereby integrate/coordinate the visual, audio and aerosol to provide a high level of immersion for the user 80. In some embodiments, the headset 82 comprises sensors which collect physiological data relating to the user (as discussed further below). In some embodiments, the headset 82 implements the control module 14 described above. The control module may be configured to select one or more active compound(s) from an actives selection module (not shown). The selected compound(s) may be provided to a heater (not shown) to generate an aerosol that is released towards the nose of the user as indicated by arrows 85. In some embodiments, the aerosol delivery mechanism 84 releases the active compound(s)/aerosol synchronously with visual and audio stimuli provided by the VR headset 82. In some embodiments, the aerosol delivery mechanism 84 releases the active compound(s)/aerosol synchronously with the data collected by the sensor(s). In some embodiments, the sensor(s), control module and heater may be part of either the headset 82 or the aerosol delivery mechanism or part of one or more separate devices or a mix thereof.

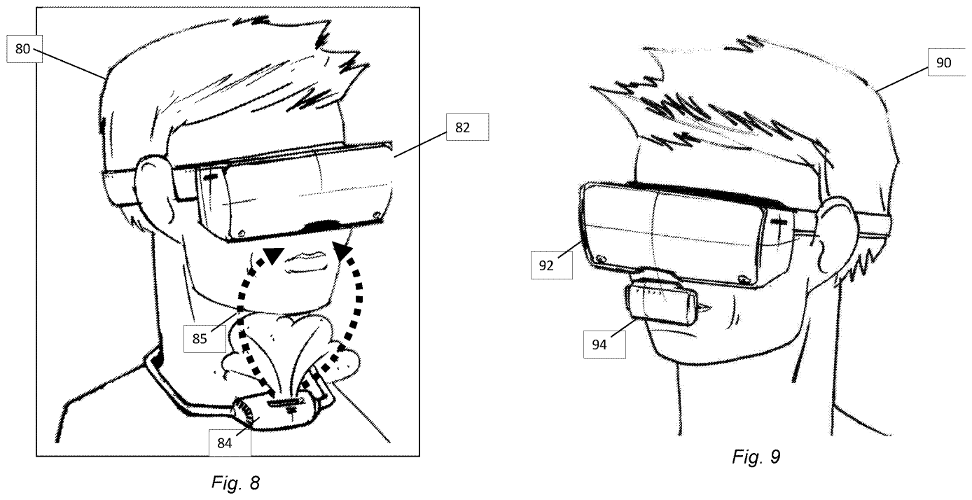

FIG. 9 shows a user 90 wearing a headset 92 (e.g. a VR, AR, MR or XR headset) and aerosol delivery mechanism 94 in accordance with an example embodiment. The aerosol delivery mechanism 94 takes the form of a mouth delivery device.

As in the system 80, the headset 92 and aerosol delivery mechanism 94 allow the user 90 to connect to a virtual world (or metaverse). The headset 92 may include some or all of the features of the headset 80 described above. In some embodiments, the aerosol delivery mechanism 94 releases the active compound(s)/aerosol synchronously with visual and audio stimuli provided by the VR headset 92. In some embodiments, the aerosol delivery mechanism 94 releases the active compound(s)/aerosol synchronously with the data collected by one or more the sensor(s). In some embodiments, the sensor(s), control module and heater may be part of either the VR headset 92 or the aerosol delivery mechanism 94 or part of one or more separate devices or a mix thereof.

In some embodiments, the aerosol delivery mechanism 94 is part, or an accessory to, the headset 94.

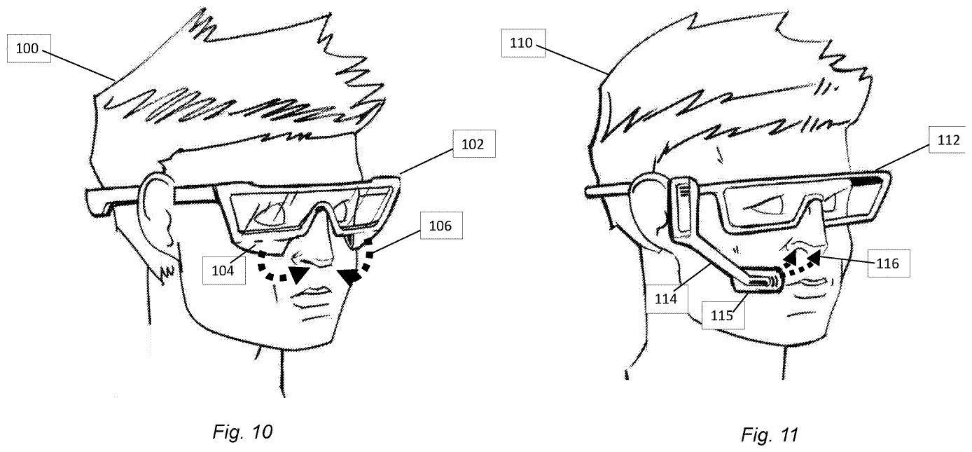

FIG. 10 shows a user 100 wearing a headset 102 (e.g. a VR, AR, MR or XR headset) and aerosol delivery mechanism 104 in accordance with an example embodiment. In use, an aerosol may be generated by the aerosol delivery mechanism 104, as indicated by the arrows 106. The aerosol delivery mechanism 104 is, or forms part of, the headset 102. As in the systems 80 and 90, the headset 102 and aerosol delivery mechanism 104 allow the user 100 to connect to a virtual world (or metaverse). The headset 102 may include some or all of the features of the headsets 80 and 90 described above.

FIG. 11 shows a user 110 wearing a headset 112 (e.g. a VR, AR, MR or XR headset) and aerosol delivery mechanism 115 in accordance with an example embodiment. In use, an aerosol may be generated by the aerosol delivery mechanism 115, as indicated by the arrows 16. In the example of FIG. 11, the aerosol delivery mechanism 115 is, or forms part of, an accessory 114 to the headset 112. As in the systems 80, 90 and 100, the headset 112 and aerosol delivery mechanism 115 allow the user 110 to connect to a virtual world (or metaverse). The headset 112 may include some or all of the features of the headsets 80, 90 and 100 described above.

In the example embodiments discussed above, the delivery mechanism is configured to be worn by the user. This is not essential to all example embodiments, as discussed further below.

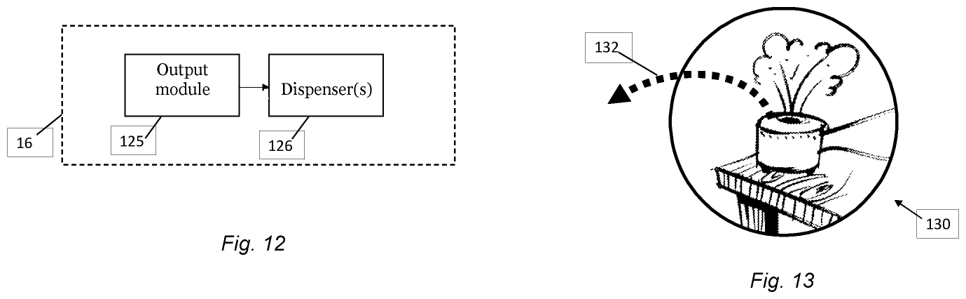

FIG. 12 is a block diagram of an example implementation of the delivery mechanism 16 of the system 10 described above, in accordance with an example embodiment. As shown in FIG. 12, the delivery mechanism 16 comprises an output module 125 (which may, in some embodiments, form part of the control module 14) and one or more dispensers 126 (which may be physically remote from the control module 14). The output module 125 may communicate (e.g. wirelessly, such as via Bluetooth®) with the one or more dispensers 126 for delivery of one or more active compounds (e.g. an aerosol(s) into a room). The one or more dispensers 126 may or may not be worn by the user.

As discussed above, the control module 14 may include a prediction module 42 for determining a future mood state or need of a user based, at least in part, on said real-time physiological data, wherein said control module determines said delivery of one or more active compounds based, at least in part, on the determined future mood state or need. Accordingly, the operation of the one or more dispensers 126 may be dependent on the predicted future mood state or need.

FIG. 13 shows an aerosol delivery mechanism, indicated generally by the reference numeral 130, in accordance with an example embodiment. The aerosol delivery mechanism 130 is an active dispenser that can deliver an aerosol (indicated by the arrow 132) into a room.

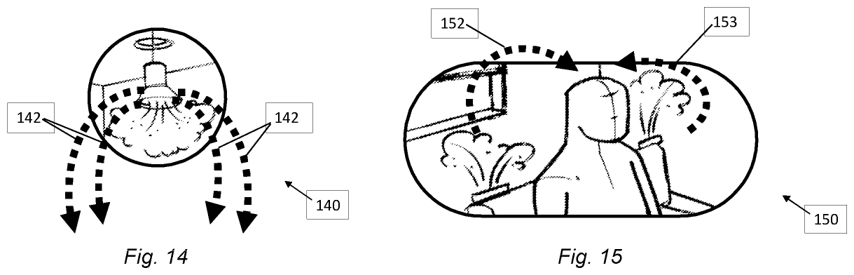

FIG. 14 shows an aerosol delivery mechanism, indicated generally by the reference numeral 140, in accordance with an example embodiment. The aerosol delivery mechanism 140 is a lightbulb or light-fitting that can deliver an aerosol (indicated by the arrows 142) into a room.

FIG. 15 shows an aerosol delivery mechanism, indicated generally by the reference numeral 150, in accordance with an example embodiment. The aerosol delivery mechanism 150 comprises a pair of active dispensers that can each deliver an aerosol (indicated by the arrows 152 and 153) into a room.

The aerosol delivery mechanisms 130, 140 and 150 are examples of the one or more dispensers 126 of the delivery mechanism 16.

The aerosol delivery mechanisms 130, 140 and 150 describe arrangements in which aerosol is delivered into a room. In some example embodiments, aerosol (or some other output, such as a mist or a spray) may be provided into multiple rooms. Thus, determined active compound(s) can be delivered to a user even if that user is moving between multiple rooms.

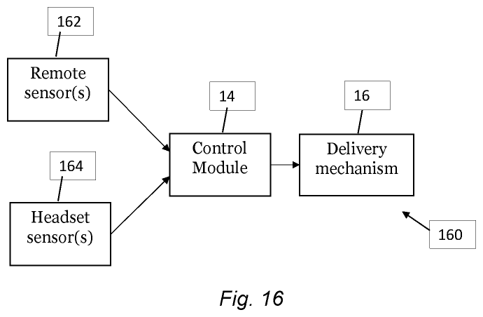

FIG. 16 is a block diagram of a system, indicated generally by the reference numeral 160, in accordance with an example embodiment. The system 160 comprises the control module 14 and the delivery mechanism 16 of the system 10 described above. The system further comprises one or more remote sensors 162 and one or more headset sensors 164. The remote sensors 162 and headset sensors 164 may collectively provide the one or more sensors 12 of the system 10 described above. It should be noted that in some example embodiments, the headset sensors may be omitted (so that only the remote sensors 162 are provided) and in other example embodiments the remote sensors may be omitted (so that only the headset sensors 164 are provided).

The control module 14 may be in communication with a remote device, such as a mobile phone or an application (a so-called “App”). The remote device may provide a user interface enabling a user to provide input and/or receive outputs from the system.

The sensor(s) described above provide real-time data (such as physiological data relating to a user) to the control module 14. The sensors may take many forms. For example, the sensors (either the remote sensors or the headset sensors) may comprise one or more imaging devices or cameras. Alternatively, or in addition, the sensors (either the remote sensors or the headset sensors) may comprise Internet of Things (IoT) devices. The sensors may form part of the delivery mechanism or part of an accessory to said delivery mechanism. Indeed, the sensors may include any sensor that transduces a physiological trait or characteristic into an electrical signal that can be processed to determine, measure or track that particular trait or characteristic. This includes sensors that are cameras, microphones, electrophysiological sensors (EEG, ECG etc.), temperature sensors etc. Sensors can also be used to detect behavioural signals such as handwriting, voice and facial characteristics. Positioning data (such as GPS data or a location within a virtual space) could be used to assess a mood state.

FIG. 17 shows a user 170 in accordance with an example embodiment. The user 170 is shown with a first data collection point 172 and a second data collection point 174 on the user's face.

FIG. 17 illustrates the parts of a human face that may be used as data collection points for one or more sensors (e.g. one or more sensors of sensors 12, 162 and 164) to provide real-time physiological data for determining a mood state of the user. The sites may include the skin areas of the forehead 172 or the cheeks 174 of the face. Example sensors include thermal sensors, non-thermal sensors, galvanic skin response (GSR) sensor, eye tracking/dilation sensors, transdermal optical imaging (TOI) sensors, and other sensors to detect brain activity etc. As discussed further below, one more sensors may be integrated into, or otherwise form part of, a headset (e.g. a virtual reality, augmented reality or extended reality headset).

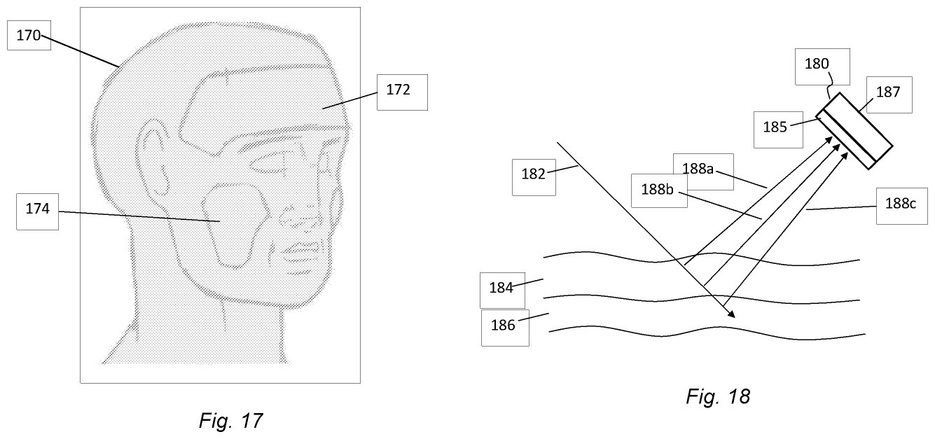

FIG. 18 shows a transdermal optical imaging (TOI) sensor 180 used in some example embodiments. By way of example, the one or more headset sensors 164 may comprise one or more TOI sensors 180.

The sensor 180 arranged to capture light reflected off/re-emitted by human skin. The skin may be parts of a human face such as skin areas 172 and 174 described above with reference to FIG. 17. In some embodiments, the sensor 180 is an optical camera configured for Transdermal Optical Imaging (TOI).

In TOI, light 182 is directed towards the skin. Part of the light 182 is absorbed in a melanin layer 184 of the skin. Part of the light 182 is absorbed in a haemoglobin layer 186 of the skin. In the example embodiment shown in FIG. 18, light of specific wavelengths (e.g. blue light) 188a is re-emitted by the melanin layer 184, light of specific wavelengths (e.g. green light) 188b is re-emitted by the melanin layer 184 and light of specific wavelengths (e.g. red light) 188c is re-emitted by the haemoglobin layer 186. The optical camera 180 may comprise an aperture 185 mounted onto a headset and arranged to capture the re-emitted blue light 188a, green light 188b and red light 188c. In some embodiments, the optical camera 180 comprises a filter (e.g. a Bayer filter) with an array of red, green and blue photosensors (not shown). In some embodiments, the optical camera 180 further comprises a processor 187 configured to process the red light captured by the red photosensors. The processor 187 may be configured to perform TOI processing to provide heart rate and heart rate variability analytics of the user. In some embodiments, the heart rate and heart rate variability analytics are provided to a prediction module (such as prediction module 42 described above) to predict a future mood state of the user.

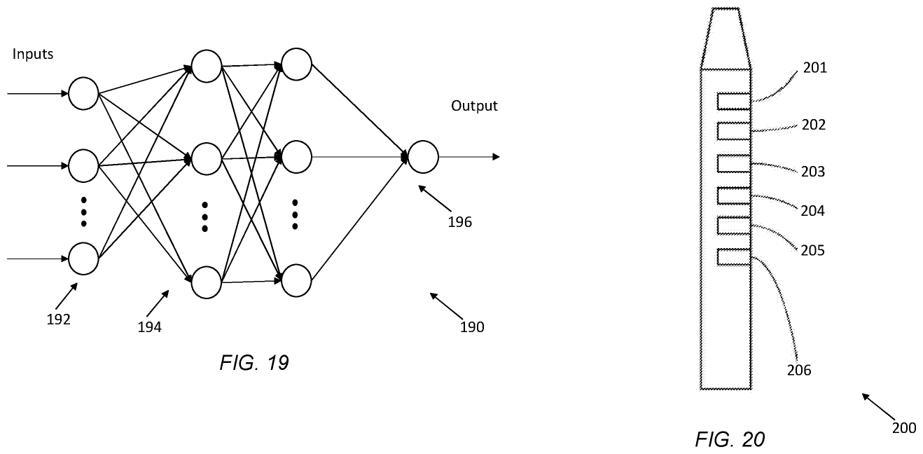

FIG. 19 is a block diagram of a neural network, indicated generally by the reference numeral 190, used in some example embodiments. For example, the prediction module 42 described above may be implemented using the neural network 190. The neural network 190 comprises an input layer 192, one or more hidden layers 194, and an output layer 196. At the input layer 192, data (such as sensor data) is received as an input. The hidden layers 194 may comprise a plurality of hidden nodes, where received sensor data are processed. At the output layer 196, one or more outputs (such as a predicted future state or need) are output.

The inputs to the model may the outputs of the one or more sensors described above. The output of the model 190 may be a future mood state or need for a user.

The model 190 may be trained based on training data of known or simulated sensor data and need states. The training may comprise re-enforcement learning or some similar technique.

As noted above, the aerosol generating device 70 is provided by way of example only. Many alternative aerosol generating devices may be used in example implementations of the principles described here. For example, the aerosol generating device 70 may have access to multiple active compounds and include a mechanism for selecting between active compounds for delivery.

FIG. 20 is a block diagram showing an aerosol delivery mechanism, indicated generally by the reference numeral 200, in accordance with an example embodiment.

In broad outline, the device 200 is configured to generate an aerosol for delivery to a user from at least one aerosolisable material received within the device 200. Herein an aerosolisable material includes any material that may be aerosolised. In the examples discussed herein the aerosol provision device 200 is configured to receive a plurality of aerosolisable materials, where each aerosolisable material is housed in or forms a consumable, e.g., the consumable may be a container housing the aerosolisable material. The aerosol provision device 200 is configured to receive at least a first consumable 201 and a second consumable 202, and the device 200 may also be configured to receive further consumables 203, 204, 205 and 206. Herein, reference is made to the device 200 receiving consumables 201 to 206; however, it should be appreciated that device 200 more generally receives a plurality of aerosolisable materials. In some implementations, the aerosolisable materials may be provided detached from one another (e.g., as separate consumables as described herein) or may be provided on a common substrate as a single consumable to be received in the device 200.

The aerosol delivery mechanism 200 may be configured to recognise the identity and position of consumables 201 to 206 received in the device 200 and may transmit data indicating the identity and position of consumables received in the device 200 (e.g. to a control module, such as the control module 14 described above). The consumables 201 to 206 may, for example, comprise radio frequency identification (RFID) tags that may be used for identification purposes.

The aerosol delivery mechanism 200 allows for a usage session which is appropriate for the consumables received within the device 200 to be implemented. Appropriate settings may be applied to the aerosol delivery mechanism 200 depending on the consumables inserted and depending on the contextual environment of the user (e.g. depending on a determined or predicted user need). Herein, reference is made to example devices transmitting data regarding the identity and position of consumables or, more generally, aerosolisable materials received in the device. It should be appreciated that in some implementations, a device may recognise the identity and/or position of consumables/materials received in the device and a controller or the like in the device may use the identity and position data to provide instructions to the device for producing an aerosol based on the identity and/or position of consumables/materials received in the device.

The device 200 comprises means for receiving at least the first consumable 201 for containing a first aerosolisable material, and for receiving the second consumable 202 for containing a second material. In some examples, the device is configured to receive further consumables, such as third 203, fourth 204, fifth 205, and sixth 206 consumables for containing third, fourth, fifth and sixth aerosolisable materials respectively. In other examples, the device 200 may be configured to receive any number, two or more, of consumables.

Aerosol is generated by the device 200 from at least the first consumable 201 containing first aerosolisable material. The first consumable 201 is in fluidic contact with a central aperture (for example via a value or flow device, not shown), and air flowing in through one or more air inlets mixes with aerosol generated from the first consumable 201 to generate a flow of aerosol. The aerosol flow is drawn towards the outlet for delivery to the user. In some examples, air flowing from the air inlets to the mouthpiece may pass through each consumable or aerosolisable material received in the device sequentially. That is, each of the consumables or aerosolisable materials in the device may be located on the same air flow path between the air inlets and the mouthpiece. In other examples, there may be multiple branches for air flowing from the air inlet/s towards the outlet. For example, a plurality of branches may be provided and each branch of the plurality of branches may pass through one or more of the consumables or aerosolisable materials. There may be one branch for each of the consumables or aerosolisable materials, or each air flow path may pass through more than one of the consumables or aerosolisable materials. In some examples, where there are multiple air flow branches there may be a branch which does not pass through a consumable or aerosolisable material. Where there are multiple air flow branches the branches may join, in an admixing chamber or the like, prior to aerosol flowing to the mouthpiece.

The second consumable 202 may also produce aerosol which mixes with the aerosol generated from the first consumable 201 before the aerosol reaches the outlet. For example, the second consumable 202 may produce a flavoured aerosol. Additionally or alternatively, one or more properties of the aerosol generated from the first consumable 201 may be modified by material contained by the second consumable 202 and, optionally, by material contained by one or more further consumables 203, 204, 205, etc. received within the device.

In some example embodiments, the aerosolisable materials may be liquids or gels; however this is not essential to all example embodiments.

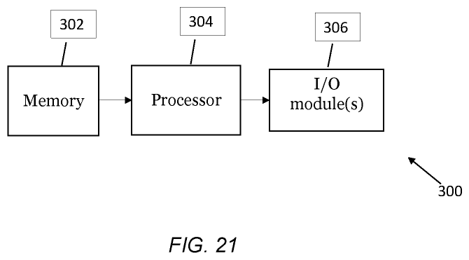

FIG. 21 is a block diagram of a processing system, indicated generally by the reference numeral 300, that may be used to implement one or more of the example embodiments described previously. The processing system 300 may, for example, be (or may include) the apparatus referred to in the claims below.

The processing system 300 may have a processor 304, a memory 302 coupled to the processor (e.g. comprising a random access memory (RAM) and/or a read only memory (ROM)). The processing system 300 may also comprise one or more input/output (I/O) modules 306, such as one or more user interface modules.

The memory 302 may comprise code which, when executed by the processor 304 implements aspects of the methods and algorithms described herein.

Aspects of the subject matter described herein are set out in the following numbered clauses:

1. A system comprising:
an input module for communicating with one or more sensors generating real-time physiological data relating to a user within a room;
a control module for determining delivery of one or more active compounds to influence, manage or control the mood state of said user; and
an output module for communicating with one or more dispensers for delivery of said one or more active compounds into the room.

2. The system according to clause 1, further comprising a prediction module for determining a future mood state or need of a user based, at least in part, on said real-time physiological data, wherein said control module determines said delivery of one or more active compounds based, at least in part, on the determined future mood state or need.

3. The system according to clause 1 or clause 2, further comprising said one or more dispensers.

4. The system according to any one of the preceding clauses, wherein said dispensers comprise one or more active dispensers.

5. The system according to any one of the preceding clauses, wherein said dispensers comprise one or more lightbulbs or light fittings.

6. The system according to any one of the preceding clauses, comprising a plurality of dispensers.

7. The system according to any one of the preceding clauses, wherein some or all of said active compounds are delivered as aerosols.

8. The system according to any one of the preceding clauses, further comprising said one or more sensors.

9. The system according to any one of the preceding clauses, wherein said sensors comprise Internet of Things (IoT) devices.

10. The system according to any one of the preceding clauses, wherein said sensors comprise imaging devices or cameras.

11. The system according to any one of the preceding clauses, wherein some or all of said sensors are remote from said user.

12. The system according to any one of the preceding clauses, wherein some or all of said sensors are in wireless communication with said system.

13. The system according to any one of the preceding clauses, wherein the user is in a virtual world.

14. The system according to any one of the preceding clauses, further comprising a headset to be worn by said user.

15. The system according to clause 14, wherein some or all of said sensors are remote from said headset.

16. The system according to clause 14, wherein some or all of said sensors are provided by the headset.

17. The system according to any one of the preceding clauses, wherein some or all of said dispensers are remote from said headset.

18. A method comprising:
communicating with one or more sensors generating real-time physiological data relating to a user within a room;
determining delivery of one or more active compounds to influence, manage or control the mood state of said user; and
communicating with one or more dispensers for delivery of said one or more active compounds into the room.

19. The method according to clause 18, further comprising determining a future mood state of a user based, at least in part, on said real-time physiological data, wherein said control module determines said delivery of one or more active compounds based, at least in part, on the determined future mood state or need.

20. The method according to clause 18 or clause 19, comprising a plurality of dispensers.

21. The method according to any one of clauses 18 to 20, wherein some or all of said active compounds are delivered as aerosols.

22. The method according to any one of clauses 18 to 21, wherein some or all of said sensors are remote from said user.

23. The method according to any one of clauses 18 to 22, wherein some or all of said sensors are in wireless communication with said system.

24. The method according to any one of clauses 18 to 23, wherein some or all of said sensors are remote from a virtual reality headset to be worn by said user.

25. The method according to any one of clauses 18 to 24, wherein the user is in a virtual world and/or wearing a headset.

26. A computer program comprising instructions for causing an apparatus to perform at least the following:
communicating with one or more sensors generating real-time physiological data relating to a user within a room;
determining delivery of one or more active compounds to influence, manage or control the mood state of said user; and
communicating with one or more dispensers for delivery of said one or more active compounds into the room.

The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilised and modifications may be made without departing from the scope of the claimed invention. Various embodiments of the invention may suitably comprise, consist of, or consist essentially of, appropriate combinations of the disclosed elements, components, features, parts, steps, means, etc., other than those specifically described herein. In addition, this disclosure may include other inventions not presently claimed, but which may be claimed in future.

1. A system comprising:
an input module for communicating with one or more sensors generating real-time physiological data relating to a user within a room;
a control module for determining delivery of one or more active compounds to influence, manage or control the mood state of said user; and
an output module for communicating with one or more dispensers for delivery of said one or more active compounds into the room.

2. The system as claimed in claim 1, further comprising a prediction module for determining a future mood state or need of a user based, at least in part, on said real-time physiological data, wherein said control module determines said delivery of one or more active compounds based, at least in part, on the determined future mood state or need.

3. The system as claimed in claim 1, further comprising said one or more dispensers.

4. The system as claimed in claim 1, wherein said dispensers comprise one or more active dispensers.

5. The system as claimed in claim 1, wherein said dispensers comprise one or more lightbulbs or light fittings.

6. (canceled)

7. The system as claimed in claim 1, wherein some or all of said active compounds are delivered as aerosols.

8. The system as claimed in claim 1, further comprising said one or more sensors.

9. The system as claimed in claim 1, wherein said sensors comprise Internet of Things (IoT) devices.

10. The system as claimed in claim 1, wherein said sensors comprise imaging devices or cameras.

11. The system as claimed in claim 1, wherein some or all of said sensors are remote from said user.

12. (canceled)

13. (canceled)

14. The system as claimed in claim 1, further comprising a headset to be worn by said user.

15. The system as claimed in claim 14, wherein some or all of said sensors are remote from said headset.

16. The system as claimed in claim 14, wherein some or all of said sensors are provided by the headset.

17. The system as claimed in claim 14, wherein some or all of said dispensers are remote from said headset.

18. A method comprising:
communicating with one or more sensors generating real-time physiological data relating to a user within a room;
determining delivery of one or more active compounds to influence, manage or control the mood state of said user; and
communicating with one or more dispensers for delivery of said one or more active compounds into the room.

19. The method as claimed in claim 18, further comprising determining a future mood state of a user based, at least in part, on said real-time physiological data, wherein said control module determines said delivery of one or more active compounds based, at least in part, on the determined future mood state or need.

20. (canceled)

21. The method as claimed in claim 18, wherein some or all of said active compounds are delivered as aerosols.

22. The method as claimed in claim 18, wherein some or all of said sensors are remote from said user.

23. (canceled)

24. The method as claimed in claim 18, wherein some or all of said sensors are remote from a virtual reality headset to be worn by said user.

25. (canceled)

26. A computer program comprising instructions for causing an apparatus to perform at least the following:
communicating with one or more sensors generating real-time physiological data relating to a user within a room;
determining delivery of one or more active compounds to influence, manage or control the mood state of said user; and
communicating with one or more dispensers for delivery of said one or more active compounds into the room.
